(19) United States
(12) Patent Application Publication
(10) Pub. No.: US 2011/0301463 A1
(43) Pub. Date: Dec. 8, 2011

(54) ULTRASONIC IMAGE DIAGNOSTIC APPARATUS

(76) Inventors: Tomokazu FUJII, Nasushiobara- (JP); Masato Oonuki, Yaita-shi (JP); Masatoshi Nishino, Otawara-shi (JP); Jiro Higuchi, Otawara-shi (JP); Osamu Nakajima, Otawara-shi (JP); Shinichi Hoshino, Otawara-shi (JP)

(21) Appl. No.: 13/154,977

(22) Filed: Jun. 7, 2011

(30) Foreign Application Priority Data
Jun. 8, 2010 (JP) 2010-131420

(51) Int. Cl. A61B 8/14

(52) U.S. Cl. 600/443

(57) ABSTRACT

According to one embodiment, an ultrasonic image diagnostic apparatus includes one or a plurality of ultrasonic probes, a scanning unit configured to scan inside an object with ultrasonic waves through the ultrasonic probe, an image generation unit configured to generate an ultrasonic image based on an output from the scanning unit, and a display unit configured to display the ultrasonic image. The ultrasonic image diagnostic apparatus further includes a posture detection unit configured to detect the posture of the display unit and a control unit configured to control the scanning unit to change a scanning condition in response to a change in the detected posture of the display unit.

### CROSS-REFERENCE TO RELATED APPLICATIONS

This application is based upon and claims the benefit of priority from Japanese Patent Application No. 2010-131420, filed Jun. 8, 2010; the entire contents of which are incorporated herein by reference.

### FIELD

Embodiments described herein relate generally to an ultrasonic image diagnostic apparatus.

### BACKGROUND

A given ultrasonic image diagnostic apparatus has a function of enlarging an image during ultrasonic diagnosis and an image display mode of simultaneously referring two or more images by using a biplane probe or the like.

Ultrasonic image diagnostic apparatuses display ultrasonic images by various methods. For example, such an apparatus simultaneously displays two images during examination. The two two-dimensional images are placed side by side in the screen. Each of the two images independently allows freeze operation and page ejection operation. This makes it possible to proceed with examination while making comparison with the currently checked region as needed. The pulse wave Doppler mode or the M mode allows a two-dimensional image and a waveform image to be juxtaposed and displayed on the left and right or upper and lower portions of the screen. Such a mode allows the operator to check the movement of an object while checking the tissue characterization of the object.

Recently, many apparatuses are provided with a panoramic view mode. In the panorama view mode, a plurality of two-dimensional images are combined into an image like a panoramic photo. The panoramic view mode can secure a field of view wider than the scanning field of view of a probe.

It is very useful to display two or more images in one window and allow to simultaneously visually check targets and compare them with each other. It is quite a common practice to simultaneously display two images in current ultrasonic diagnosis. The display size of each of images to be simultaneously displayed is inevitably smaller than that of an image to be singly displayed. This leads to a deterioration in visibility. When, for example, two images are to be simultaneously displayed, the images are placed side by side. For this reason, the resolution of the display screen in the longitudinal direction is the same as that of a two-dimensional image in the single image display mode. However, the width in the lateral direction theoretically decreases to half, because the two images are juxtaposed. That is, a horizontally long organ or object, which can be fit in one window in the single image display mode, may not be fit in one window in the two-image simultaneous display mode. Alternatively, such an observation target needs to be adjusted so as to be fit in the screen by changing the reduction scale in the screen. Information loss (deterioration in visibility) inevitably occurs as compared with a two-dimensional image in the single image display mode. The same applies to the pulse wave Doppler mode and the M mode. In the pulse wave Doppler mode, in particular, when simultaneously displaying a two-dimensional image and a waveform, the operator usually designates a place where he/she wants to acquire a Doppler waveform on the two-dimensional image, and a waveform at the designated portion is then displayed on a lower portion of the screen, on the left of the two-dimensional image, or the like. This inevitably narrows the display range. In order to check the properties of a two-dimensional image to a certain degree, the display operation is similar to that in the two-image simultaneous display mode. In this case, however, the Doppler waveform display range is narrow. When checking a Doppler waveform, while a waveform pattern is repeatedly acquired to a certain degree, the shape and the like of the waveform itself are measured. The “narrowness” will lead to an undesirable state in terms of examination.

As a solution proposal for this problem, a method of vertically juxtaposing a two-dimensional image and a Doppler waveform is available. In this case, however, the two-dimensional image is obviously displayed in a reduced scale, which is not suitable to check tissue characterization while watching the Doppler waveform. Although conventional apparatuses have improved this problem by displaying a two-dimensional image and a Doppler waveform upon changing the size ratio to a certain degree, one of the images is inevitably reduced in size (or width). That is, this technique works only in the direction of decreasing accuracy in examination.

In addition to the above technique, some ultrasonic image diagnostic apparatuses use a probe called a biplane probe, in a body cavity and the like, which is provided with two heads configured to perform transmission/reception. Such an apparatus generally uses the two-image simultaneous display mode as a display form, and independently displays images obtained by the two heads on the left and right sides.

In general, however, the heads of the biplane probe are conceptually juxtaposed vertically instead of horizontally. For this reason, the arrangement of actually captured images does not coincide with that in the screen. Furthermore, this biplane probe is used as a transrectal/transvaginal probe. That is, display in the two-image simultaneous display mode is not intuitive display also from the viewpoint of the usage method of the probe.

There is a recent technique that generates two-dimensional images (still images) in ranges much wider than the scanning field of view of a probe by superimposing the two-dimensional images while shifting them within a plane. This technique is used for peripheral vascular examination and the like.

A panoramic view mode technique is especially effective for extremity examination. A plurality of images are pasted to each other to display considerably long (wide) extremities. For this reason, when trying to display an entire image of the extremities as large as possible within the screen, the long axis is made to match the lateral direction of the screen. At this time, in general, in consideration of the positional relationship between the ultrasonic image diagnostic apparatus and the bed, the direction in which the bed and the probe are thought to be scanned is perpendicular to the lateral direction of the screen of the ultrasonic diagnostic apparatus. That is, this direction does not coincide with an intuitive direction.

### BRIEF DESCRIPTION OF THE DRAWINGS

FIG. 1 is a block diagram showing the arrangement of an ultrasonic image diagnostic apparatus according to an embodiment;

FIG. 2 is a flowchart showing a procedure for performing screen display switching processing under the control of a control unit in FIG. 1;

FIG. 3 is a view showing screen display switching performed in response to display screen rotation during the use of the two-dimensional mode under the control of the control unit in FIG. 1;

FIG. 4 is a view showing screen display switching and scanning condition (scanning depth of field) switching performed in response to display screen rotation during the use of the two-dimensional mode under the control of the control unit in FIG. 1;

FIG. 5 is a view showing screen display switching performed in response to display screen rotation during the use of the two-image simultaneous display mode under the control of the control unit in FIG. 1;

FIG. 6 is a view showing screen display switching performed in response to display screen rotation in the pulse waveform Doppler mode under the control of the control unit in FIG. 1;

FIG. 7 is a view showing screen display switching performed in response to display screen rotation in the report mode under the control of the control unit in FIG. 1;

FIG. 8 is a view showing screen display switching performed in response to display screen rotation during the use of a biplane probe under the control of the control unit in FIG. 1;

FIG. 9 is a view showing screen display switching performed in response to display screen rotation during the use of a three-dimensional scanning probe under the control of the control unit in FIG. 1; and

FIG. 10 is a view showing screen display switching performed in response to display screen rotation during the use of the three-dimensional scanning probe under the control of the control unit in FIG. 1.

### DETAILED DESCRIPTION

In general, according to one embodiment, an ultrasonic image diagnostic apparatus includes one or a plurality of ultrasonic probes, a scanning unit configured to scan inside an object with ultrasonic waves through the ultrasonic probe, an image generation unit configured to generate an ultrasonic image based on an output from the scanning unit, and a display unit configured to display the ultrasonic image. The ultrasonic image diagnostic apparatus further includes a posture detection unit configured to detect the posture of the display unit and a control unit configured to control the scanning unit to change a scanning condition in response to a change in the detected posture of the display unit.

An embodiment of the present invention will be described below with reference to the views of the accompanying drawing.

For the general screen structure of a display device, i.e., an aspect ratio of 3:4, it is preferable to effectively use the amount of information of an image as a display target by displaying the image in the maximum range in the screen. For this purpose, in this embodiment, the aspect ratio is changed by rotating the screen. On the other hand, in some cases, in order to suitably display images, it is preferable not to always rotate the screen when displaying the images. In the embodiment, when the observer determines that vertical/horizontal rotation of the screen is required, the vertical/horizontal rotation of the screen is executed. In addition, the operation by the observer which is required for the determination is facilitated to suppress a deterioration in operability.

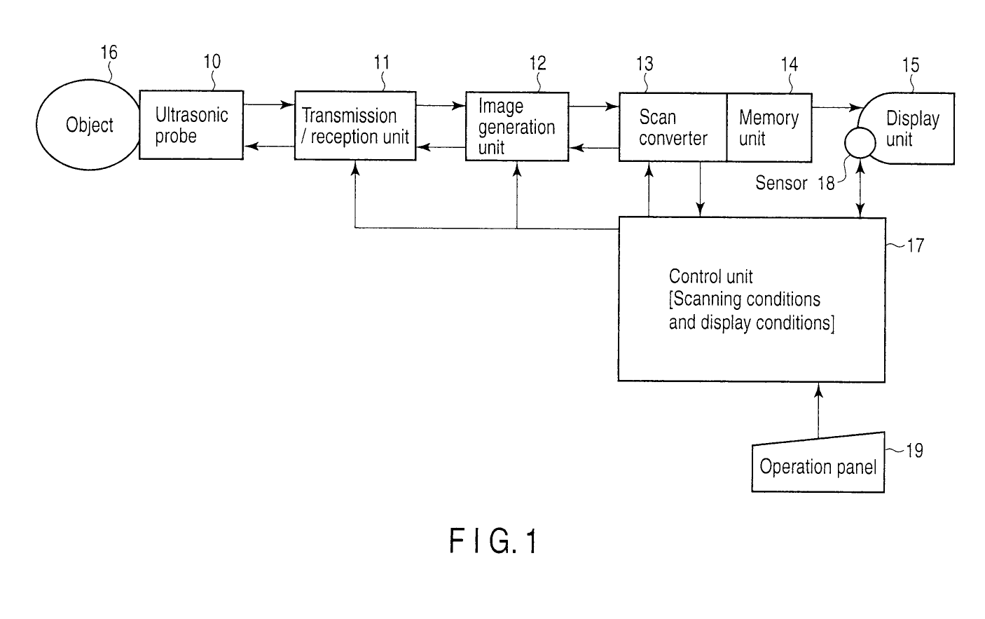

FIG. 1 is a block diagram showing the arrangement of the ultrasonic image diagnostic apparatus according to this embodiment. At least one ultrasonic probe 10 to be brought into contact with an object 16 is detachably connected to the ultrasonic image diagnostic apparatus according to this embodiment. The ultrasonic probe 10 is typically of a one-dimensional array type that has a plurality of transducers arrayed in a line in an azimuth direction. However, this probe may be of a two-dimensional array type that has a plurality of transducers arrayed two-dimensionally, and a body cavity probe having a special shape to be inserted into a body cavity, or the like. Many body cavity probes have two transducer arrays arranged at the distal end portion such that the scanning surfaces are perpendicular to each other.

A transmission/reception unit 11 transmits ultrasonic waves to the object 16 by driving the ultrasonic probe 10. The transmission/reception unit 11 receives echoes from the object 16 via the ultrasonic probe 10 and generates an electrical reflection signal. The transmission/reception unit 11 scans an ultrasonic scanning surface with an ultrasonic beam by controlling transmission/reception delays. An image generation unit 12 generates an ultrasonic image based on the reception signal output from the transmission/reception unit 11. An ultrasonic image is a B-mode image, an M-mode image, a color Doppler image, a pulse wave Doppler image, a continuous wave Doppler image, or an image obtained by combining two arbitrary types of modes of them. A scan converter 13 combines an ultrasonic image with scanning conditions including patient information and image quality conditions to generate a display image corresponding to the display screen of a display unit 15 under the control of a control unit 17. The display unit 15 displays the display image via a memory unit 14.

The display unit 15 is provided with the display screen so as to be vertically and horizontally rotatable. Typically, the display unit 15 is a portable display connected to the main body of the ultrasonic image diagnostic apparatus via a flexible cable. However, the display unit 15 may be configured to be supported on a support base so as to allow to perform vertical/horizontal rotation of the display screen portion. A sensor 18 which detects the posture of a display screen is mounted on the display unit 15. The sensor 18 detects posture data necessary for the control unit 17 to discriminate whether the display screen is in a vertical posture in which the longitudinal direction of the display screen is almost parallel to the vertical direction upon vertical/horizontal rotation by the operator or in a horizontal posture in which the longitudinal direction of the display screen is almost parallel to the horizontal direction upon vertical/horizontal rotation by the operator. For example, the sensor 18 outputs a signal reflecting the tilt angle of the display screen relative to a reference position, with the position of the display screen being a reference (0°) when the display screen is in a horizontal posture in which the longitudinal direction of the display screen is almost parallel to the horizontal direction. The control unit 17 recognizes in accordance with an output from the sensor 18 that the display screen is in a vertical posture, when, for example, the rotational angle of the display screen falls 45° relative to the reference position, which is 90°, i.e., falls within the range of 45° to 135°. Likewise, the control unit 17 recognizes in accordance with an output from the sensor 18 that the display screen is in a horizontal posture, when, for example, the tilt angle of the display screen falls within an angular range other than the range of 45° to 135° relative to the reference position.

An operation unit 19 operated by the operator is connected to the control unit 17. The operator can set in advance (preset), via the operation unit 19, scanning conditions and display conditions to be changed in accordance with the rotation of the display screen of the display unit 15 typically when the display screen is rotated from a horizontal posture to a vertical posture. The scanning conditions include a scanning resolution (the number of scanning lines per unit angle and a sampling pitch in the distance direction), a view angle (the angle or distance between an ultrasonic scanning line at one end of the scanning surface and an ultrasonic scanning line at the other end), a depth of field, the B mode/M mode/Doppler mode/color Doppler mode/operation mode combining two of them, and a change of a plurality of types of probes 10 connected to the apparatus main body. The display conditions include the direction of the erect position of a display image relative to the longitudinal direction of the display screen, the transition angle of a vertical/horizontal posture change, the position and range of an ultrasonic image relative to the display screen, patient information to be superimposed on the image, the position and range of a scanning condition including an image quality condition, and a screen layout associated with the array of two ultrasonic images or two kinds of ultrasonic images in a combinational operation mode.

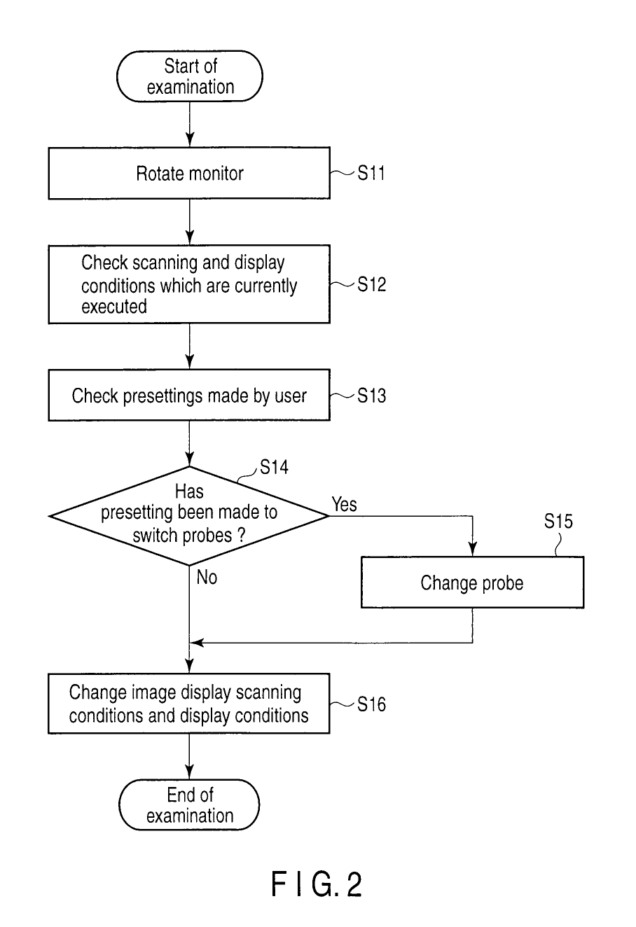

In this embodiment, when the display screen of the display unit 15 is rotated from a horizontal posture to a vertical posture, scanning conditions and display conditions are automatically changed accordingly. This operation will be described with reference to FIG. 2. When the operator rotates the display unit 15 from a horizontal posture to a vertical posture, the control unit 17 recognizes the rotation of the display screen (posture change) based on an output from the sensor 18 (S11). The control unit 17 checks the scanning conditions and display conditions which are currently executed (S12). The control unit 17 also checks the scanning conditions and display conditions which are preset with respect to a posture change from a horizontal posture to a vertical posture (S13). In order to change the scanning conditions which are currently executed to the preset scanning conditions, the control unit 17 determines whether it is necessary to change the probe (S14). If it is necessary to change the probe, the control unit 17 controls switching of a connector which connects the probe 10 to the apparatus main body, or displays a message to prompt the operator to replace the probe 10 by another type of probe 10 (S15).

The control unit 17 controls the transmission/reception unit 11, the image generation unit 12, and the scan converter 13 to execute changes to the preset scanning conditions and display conditions. The changes to the preset scanning conditions and display conditions will be described below with reference to a concrete case.

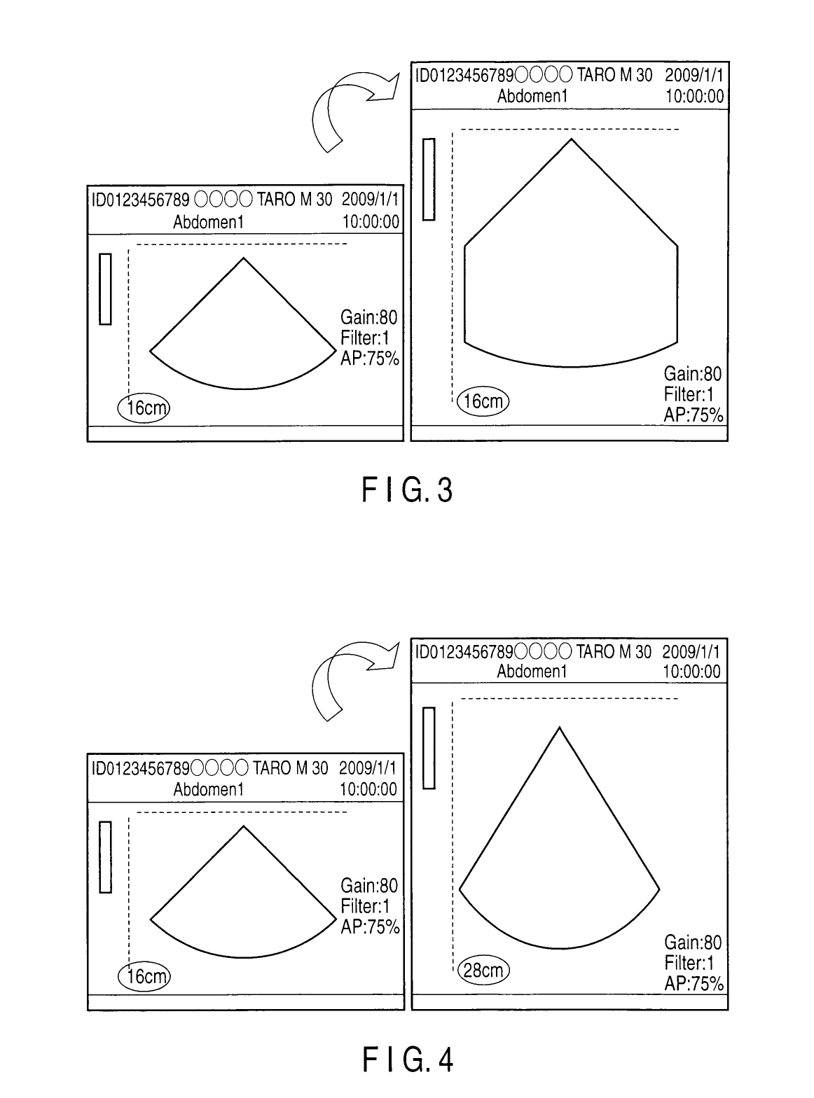

Upon determining a posture change based on an output from the sensor 18, the control unit 17 checks the current display mode of the ultrasonic image diagnostic apparatus (the two-dimensional mode (B mode)/two-image simultaneous display mode/pulse wave Doppler mode/M mode). If the current display mode is the two-dimensional mode, it is thought that the operator wants to see the two-dimensional image displayed in the screen in a larger scale (with a higher display resolution) or as an image in the depth direction (at a larger depth of field). With regard to this selection, the user can make settings in advance, e.g., presetting operation in the ultrasonic apparatus. The control unit 17 changes the display of the two-dimensional image in accordance with the settings. That is, if the user wants to see an image in a larger size, the control unit 17 displays the image upon enlarging the image so as to maintain the scanning depth of field in the longitudinal direction and cutting (trimming) image portions in the lateral direction which fall outside the screen. Since the aspect ratio and the screen display direction inevitably change, the control unit 17 adjusts the position and size of a banner portion that displays patient information or auto data and the like indicating image quality settings so as to make them fall within the screen (FIG. 3).

If the presetting has been made to see an image broad in the depth direction, the control unit 17 decreases the view angle (azimuth angle) without changing the screen enlargement ratio so as to display the image up to a scanning depth of field that allows the image to fall within the screen in the longitudinal direction (FIG. 4). A banner portion displaying patient information and an automatic data portion displaying scanning conditions and the like behave in the same manner as in the case in which the presetting has been made for enlarged display.

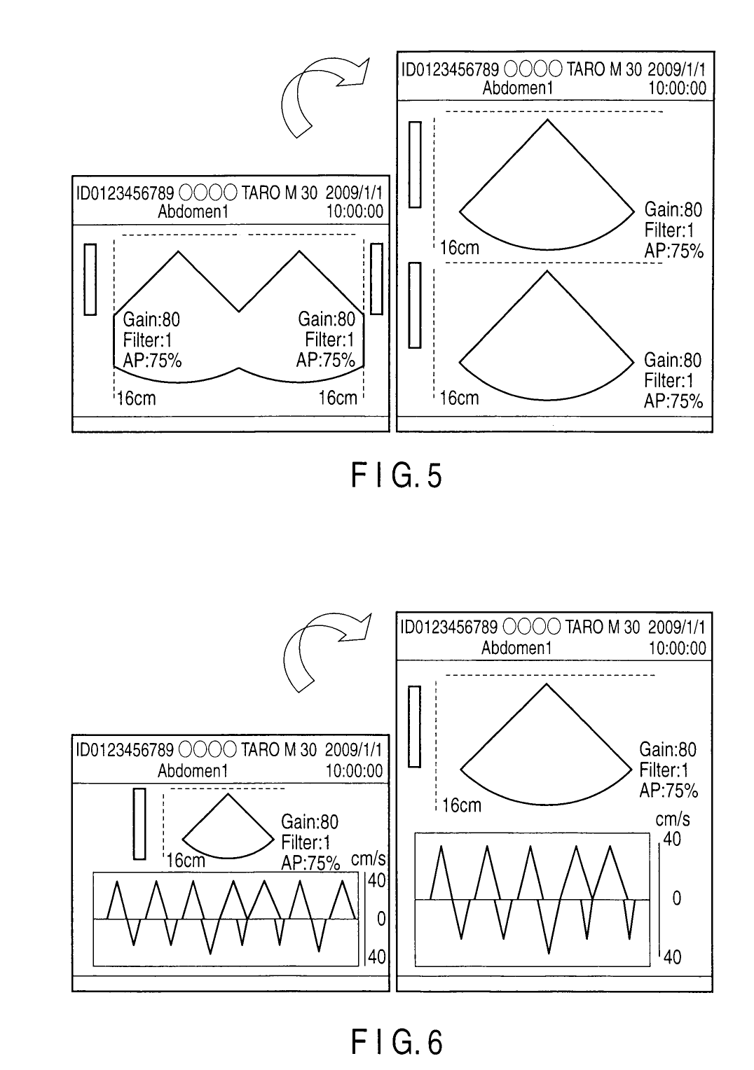

When the user rotates the display screen in the two-image simultaneous display mode using the probe 10, the control unit 17 determines that the user wants to display the two images on the upper and lower sides, and displays the two images upon vertically juxtaposing them (FIG. 5). In this case, the scanning depth of field is changed to be smaller than in the normal display mode (horizontal posture). However, the images are displayed broad in the lateral direction (azimuth angle). If examination targets are observation targets vertically juxtaposed on an object, the positional relationship with the object coincides with screen display.

Both the pulse wave Doppler mode and the M mode are expected to allow the user to simultaneously see a two-dimensional image and a waveform image (which is called this way for the convenience sake, even though it corresponds to a Doppler waveform in the pulse wave Doppler mode and to a graph representing the motion of an organ as a function of time instead of a waveform in a strict sense in the M mode, because they both appear as waveforms). When the user rotates the display screen in such a mode, the control unit 17 determines that the user wants to measure the shape of a waveform or perform short-time measurement while watching a two-dimensional image of an organ in a large size, although the waveform image need not be very long in the time direction. The control unit 17 then displays the two-dimensional image (B-mode image or color Doppler image) on the upper side of the screen and the waveform image on the lower side of the screen (FIG. 6). At this time, the two-dimensional image is displayed larger than in the pulse wave Doppler mode in normal display operation. Assume that the user presets the interpretation of the word “larger” as in the case of display screen rotation in the two-dimensional display mode, and can select either the mode of enlarging the image in a pure sense or the mode with priority on scanning depth of field.

In addition to the general two-dimensional mode/two-image simultaneous display mode/pulse wave Doppler mode/M mode, recent ultrasonic image diagnostic apparatuses have a three-dimensional display mode and special image display modes based on various types of parameter analyses. It is also conceivable to set, for example, a mode of displaying an image of interest (e.g., a volume image or important slice in three-dimensional display) in a large size upon display screen rotation as in conventional image modes such as the two-dimensional mode.

In addition, in general ultrasonic diagnosis, for example, measurement is performed on images to determine the size and thickness of a tumor or blood vessel wall, or examination is performed in perinatal medical treatment by graphing the growth of a fetus and the like. In this case, the number of times of execution of measurement and the amount of information to be displayed vary depending on the examination to be performed. For this reason, for example, information about an examination with a large amount of information may not be sufficiently displayed on a report window displaying the examination result.

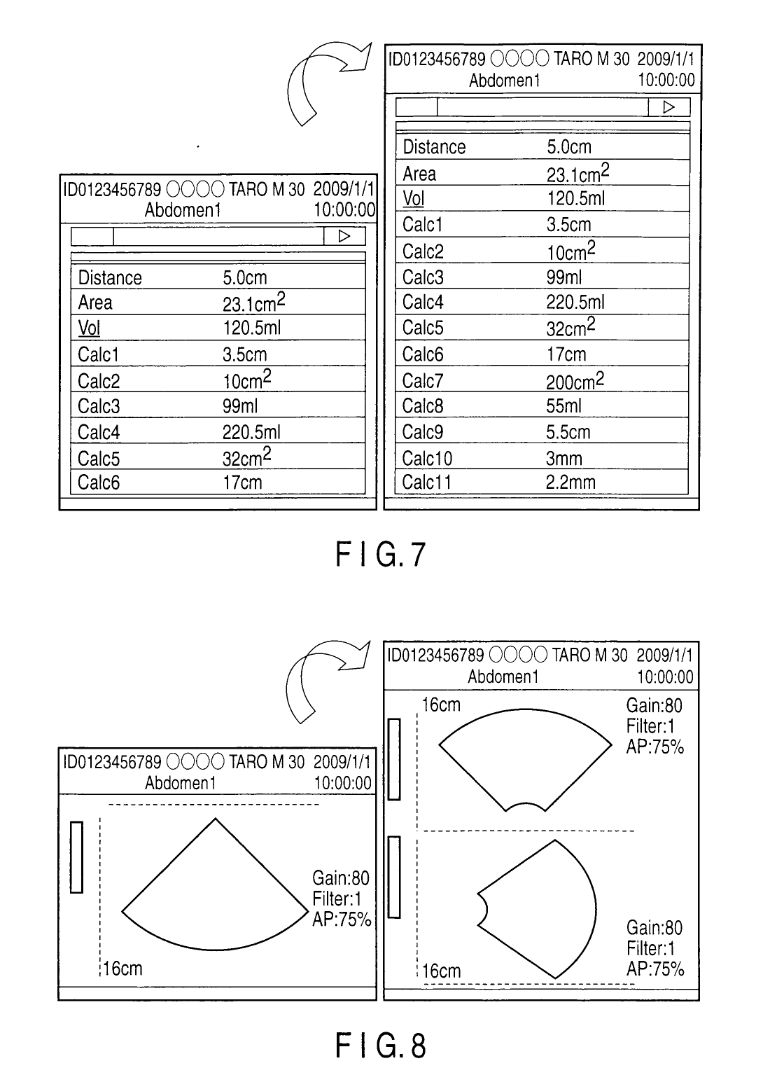

Conventionally, in this case, such information is displayed in the page flipping mode. In consideration of the efficiency of doctor's determination and the like, it is preferable to increase the amount of information that can be displayed at once. In general, of measurement data, a measurement name and result are sequentially displayed. For this reason, the display data do not relatively spread in the lateral direction but the items of the data are juxtaposed in the longitudinal direction. In terms of display, therefore, the larger the display area in the longitudinal direction, the larger the amount of information displayed in one window. Although it is possible to increase the amount of information displayed at once by decreasing the display size of each character, the visibility deteriorates. For this reason, as an application of this embodiment, when displaying a long report, it is conceivable to increase the number of items that can be displayed at once, without reducing display characters, to improve the examination efficiency by rotating the monitor (FIG. 7). Likewise, although a report is generally printed as a vertically long image, since the print image is difficult to display on the screen, the display screen rotation technique can be applied to check the print image.

The following is a case in which scanning conditions are changed in response to display screen rotation. In general, it is possible to connect a plurality of types of probes to an ultrasonic image diagnostic apparatus. An examiner generally switches and uses such probes in accordance with examination areas of objects and situations. There are different types of probes suitable for ultrasonic examinations at deep and shallow levels in the body of an object. If, for example, it is determined that the user wants to display an image up to a deep portion upon display screen rotation, it is possible to improve the examination efficiency by automatically switching to one of the probes connected to the ultrasonic image diagnostic apparatus which is suitable for examination at a deep level. In addition, the probes include a biplane probe which has two heads mounted on it and can simultaneously acquire images in two directions. In general, this biplane probe has one head mounted on the distal end and the other head mounted on a side surface. The display form of this probe is most similar to the usage state when vertically juxtaposing screen images. When, therefore, a biplane probe is connected to the ultrasonic image diagnostic apparatus upon display screen rotation, it is conceivable to automatically switch to this display form and set a vertical two-image simultaneous display mode layout (in some case, a lower image indicating a side surface image obtained by the biplane probe is rotated laterally through 90°), as shown in FIG. 8.

Note that the switching function is executed in order of priority. Assume that a body cavity probe corresponding to a biplane probe and a general probe corresponding to a single plane probe are simultaneously connected to the apparatus. In this case, when the display screen is rotated during two-dimensional examination using the general probe, the effect changes depending on whether the screen display is enlarged (or a large scanning depth of field is secured) while the currently executed two-dimensional display is maintained or the probe is switched to the biplane probe. In such a case, therefore, the user presets priority levels to the respective operation modes to make the apparatus operate in accordance with the operating environment.

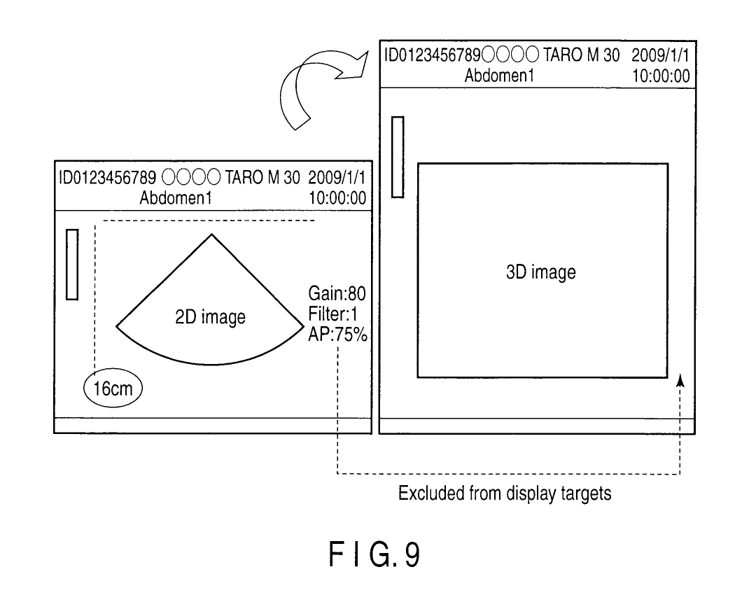

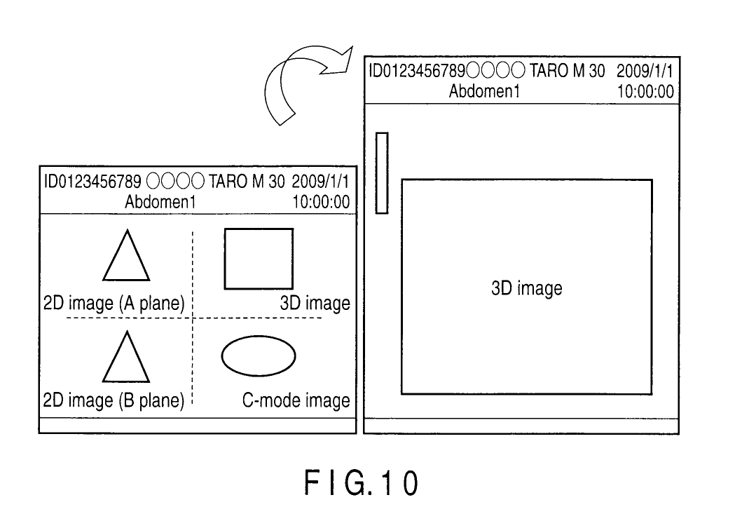

FIGS. 9 and 10 each show an example of how screen display switching is performed when a three-dimensional scanning probe is used as the probe 10. The three-dimensional scanning probe can move the direction of scanning lines to two directions, namely an azimuth direction and a plane direction perpendicular to the azimuth direction, by phase control. This implements three-dimensional scanning. This probe repeats three-dimensional scanning in a predetermined cycle. With this operation, the image generation unit 12 repeatedly generates volume data corresponding to the tissue structure of a three-dimensional region in an object.

When the display unit 15 is in a horizontal posture, the image generation unit 12 generates a two-dimensional image associated with arbitrarily one or three slices from volume data under the control of the control unit 17. The display unit 15 then display the image. When the display unit 15 is in a vertical posture, the image generation unit 12 generates a three-dimensional image of an arbitrary organ by two-dimensional conversion processing such as rendering from volume data under the control of the control unit 17.

Two-dimensional image display is mainly intended to support diagnosis by a doctor. In contrast, three-dimensional image display is mainly intended to help a doctor to explain a diagnosis result and the like to a patient. For this purpose, in order to allow a recumbent patient to easily see the display, it is preferable to switch the display image to a three-dimensional image when, for example, the display unit 15 in a horizontal position, i.e., a horizontal posture, tilts through 5° or more. In this manner, it is preferable to arbitrarily preset transition angles as changes in vertical/horizontal posture in accordance with display purposes and environments. In consideration of the purpose of three-dimensional image display, signal processing parameters such as a gain and filter type are excluded from the display screen.

As has been described above, this embodiment can easily switch screen display and the probe to be used and improve the examination throughput by combining the rotation of the monitor and the general functions of the ultrasonic image diagnostic apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic image diagnostic apparatus comprising:
one or a plurality of ultrasonic probes;
a scanning unit configured to scan an inside of an object with ultrasonic waves through the ultrasonic probe;
an image generation unit configured to generate an ultrasonic image based on an output from the scanning unit;
a display unit configured to display the ultrasonic image;
a posture detection unit configured to detect a posture of the display unit; and
a control unit configured to control the scanning unit to change a scanning condition in response to a change in the detected posture of the display unit.

2. The apparatus of claim 1, wherein the control unit changes at least one of a resolution, a view angle, a depth of field, and an operation mode combining two of a B mode, an M mode, a Doppler mode, and a color Doppler mode.

3. The apparatus of claim 1, wherein the control unit changes connection of one of the plurality of ultrasonic probes to the scanning unit.

4. The apparatus of claim 1, wherein the ultrasonic probe includes an arrangement for scanning two intersecting planes, and
the control unit changes one of the two intersecting planes to the other plane as a scanning plane.

5. The apparatus of claim 1, wherein the display unit comprises a portable display.

6. The apparatus of claim 1, wherein a screen layout of the display unit is changed together with the scanning condition.

7. The apparatus of claim 1, wherein when the display unit is placed in a vertical posture, a scanning view angle is decreased and a depth of field is increased as compared when the display unit is placed in a horizontal posture.

8. The apparatus of claim 1, wherein one slice is scanned when the display unit is placed in a horizontal posture, and two slices are scanned when the display unit is placed in a vertical posture.

9. The apparatus of claim 1, wherein when the display unit is placed in a vertical posture, a scanning resolution is higher than a case in which the display unit is placed in a horizontal posture.

10. The apparatus of claim 1, wherein a change of posture of the display unit in response to which the scanning condition is changed is arbitrarily set.

11. An ultrasonic image diagnostic apparatus comprising:
one or a plurality of ultrasonic probes;
a scanning unit configured to scan an inside of an object with ultrasonic waves through the ultrasonic probe;
an image generation unit configured to generate one or a plurality of ultrasonic images based on an output from the scanning unit;
a display unit configured to display the ultrasonic image;
a posture detection unit configured to detect a posture of the display unit; and
a control unit configured to control the display unit to change a display layout of the display unit in response to a change in the detected posture of the display unit.

12. The apparatus of claim 11, wherein the display unit comprises a portable display.

13. The apparatus of claim 11, wherein when the display unit is placed in a vertical posture, a display enlargement ratio of the ultrasonic image is higher than a case in which the display unit is placed in a horizontal posture.

14. The apparatus of claim 11, wherein the ultrasonic image is entirely displayed when the display unit is placed in a horizontal posture, and the ultrasonic image is displayed while part of the image is trimmed when the display unit is placed in a vertical posture.

15. The apparatus of claim 11, wherein the plurality of ultrasonic images are juxtaposed in a lateral direction when the display unit is placed in a horizontal posture, and the plurality of ultrasonic images are juxtaposed in a longitudinal direction when the display unit is placed in a vertical posture.

16. The apparatus of claim 15, wherein the plurality of ultrasonic images have intersecting scanning planes.

17. The apparatus of claim 11, wherein the ultrasonic image is displayed as a two-dimensional image when the display unit is placed in a horizontal posture, and the ultrasonic image is displayed as a three-dimensional image when the display unit is placed in a vertical posture.

18. An ultrasonic image diagnostic apparatus comprising:
one or a plurality of ultrasonic probes;
a scanning unit configured to scan an inside of an object with ultrasonic waves through the ultrasonic probe;
an image generation unit configured to generate an ultrasonic image based on an output from the scanning unit;
a display unit configured to display the ultrasonic image;
a posture detection unit configured to detect a posture of the display unit; and
a control unit configured to control the scanning unit and the display unit to change a scanning condition and a display layout in response to a change in the detected posture of the display unit.
